(19) United States
(12) Patent Application Publication
(10) Pub. No.: US 2026/0174960 A1
(43) Pub. Date: Jun. 25, 2026

(54) AUTOMATED INSULIN DELIVERY SYSTEM USING PRAMLINTIDE

(71) Applicant: INSULET CORPORATION, Acton, MA (US)

(72) Inventors: Joon Bok LEE, Acton, MA (US); Jason O'CONNOR, Acton, MA (US); Yibin ZHENG, Hartland, WI (US); Ashutosh ZADE, San Diego, CA (US)

(21) Appl. No.: 19/536,920

(22) Filed: Feb. 11, 2026

Related U.S. Application Data
(62) Division of application No. 17/582,374, filed on Jan. 24, 2022, now Pat. No. 12,551,619.
(60) Provisional application No. 63/143,437, filed on Jan. 29, 2021.

(51) Int. Cl. A61M 5/172; A61B 5/00; A61B 5/145; A61K 38/22; A61K 38/26; A61K 38/28; A61M 5/14; A61M 5/142; G16H 20/17; G16H 40/67

(52) U.S. Cl. CPC A61M 5/1723; A61B 5/14532; A61B 5/4839; A61B 5/4866; A61B 5/7282; A61K 38/22; A61K 38/26; A61K 38/28; A61M 5/1408; A61M 5/14248; G16H 20/17; G16H 40/67; A61M 2205/502; A61M 2205/52; A61M 2205/8206; A61M 2230/201

(57) ABSTRACT

Disclosed herein are systems and methods for the delivery of insulin and pramlintide using an automated insulin delivery system. In a first embodiment, a drug delivery system is configured to deliver independent doses of insulin and pramlintide. The system monitors the user's blood glucose level and determines when a meal is been ingested and, in response, delivers the dose of pramlintide which, in turn alters the required delivery of insulin. In the second embodiment, the drug delivery system is configured to deliver a co-formulation of insulin and pramlintide as basal doses. The total amount of pramlintide delivered in a most recent pre-determine period of time, for example, 24 hours, is used to alter the aggressiveness of the algorithm which determines the basal doses of the co-formulation.

### RELATED APPLICATIONS

This application is a division of U.S. patent application Ser. No. 17/582,374, filed Jan. 24, 2022, which claims the benefit of U.S. Provisional Patent Application No. 63/143,437, filed Jan. 29, 2021, the contents of which are incorporated herein by reference in their entirety.

### BACKGROUND

Pramlintide is an analog to the naturally occurring human hormone Amylin, whose primary effect is reduced gastric emptying, and thus decreased acute impact of long-term insulin needs. Pramlintide is known to reduce the peak, but increase the breadth, of post-prandial hyperglycemia in people with type I diabetes, due to the resulting delayed gastric emptying.

While there is not a specific dosage that is recommended for a specified meal ingestion, there is a fixed dosage recommended by clinicians for a fixed outcome in the user's glucose excursions. A clinical study indicated that over a long term, a Pramlintide treatment of 30-60 μg/meal led to a reduction in AIC by 0.5%, which translates to a mean glucose reduction of 14 mg/dL. Given the typical target glucose of 120 mg/dL, this can be considered an approximately 11.7% reduction in the user's insulin needs.

Many conventional automatic drug delivery (AID) systems are well known, including, for example, drug delivery devices of the type shown in FIG. 1 as reference number 102. The drug delivery device can be designed to deliver any type of liquid drug to a user. In specific embodiments, the drug delivery device can be, for example, an OmniPod@drug delivery device manufactured by Insulet Corporation of Acton, Massachusetts. The drug delivery device can be a drug delivery device such as those described in U.S. Pat. Nos. 7,303,549, 7,137,964, or U.S. Pat. No. 6,740,059, each of which is incorporated herein by reference in its entirety.

AID systems typically require user interaction to compensate for the largest disturbances to glucose control in people with diabetes, typically due to the ingestion of meals resulting in the absorption of fast-acting carbohydrates. This is due to the limited aggressiveness with which the AID system can act against minor deviations in glucose, leading to the user experiencing an extended period of hyperglycemia if there are no manual compensations (e.g., manually-administered bolus doses) for meals. Pramlintide slows absorption of meals and allows less aggressive insulin delivery action by AID systems to “catch up” and sufficiently compensate for meals without requiring user interactions.

Therefore, it would be desirable to enhance current AID systems to deliver Pramlintide and account for the effect of Pramlintide on the user's glucose levels and the ability of the AID to effectively control the user's glucose level, particularly during the post-prandial period.

### Definitions

As used herein, the term “liquid drug” should be interpreted to include any drug in liquid form capable of being administered by a drug delivery device via a subcutaneous cannula, including, for example, insulin, morphine, blood pressure medicines, chemotherapy drugs, fertility drugs or the like or co-formulations of two or more of GLP-1, pramlintide, and insulin.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Embodiments of the invention disclosed herein include various ways of combining the delivery of Pramlintide with the delivery of insulin to provide better control of the user's glucose excursions during the post-prandial period.

In a first embodiment of the invention, the drug delivery device is provided with two reservoirs, one containing Pramlintide and one containing insulin. When the system detects that the user has ingested a meal, the user is injected with a dose of Pramlintide to slow the absorption of the meal. Because the medication delivery algorithm limits the aggressiveness with which the algorithm can act against minor deviations in glucose, slowing the absorption of the meal will allow the less aggressive insulin delivery action determined by the medication delivery algorithm to “catch up” and sufficiently compensate for meals without requiring user interactions.

In a second embodiment of the invention, the drug delivery device is provided with a single reservoir containing a co-formulation of insulin and Pramlintide, such that the basal delivery of insulin also includes a basal delivery of Pramlintide. In this embodiment, the delivery of the Pramlintide is incorporated into the medication delivery algorithm, allowing an increase in the aggressiveness of the algorithm in acting against minor glucose excursions, given the reduced concern of extreme hyperglycemia occurring (which would result in increased risk of over delivery of insulin). In addition, the insulin constraint of the medication delivery algorithm may be reduced, given the extended period of each hyperglycemic event.

While exemplary embodiments of the present disclosure refer to a co-formulation of insulin and pramlintide, other co-formulations or drug combinations may be used. For example, a co-formulation of insulin and GLP-1 may be used; or a co-formulation of insulin and other types of glucagon-like peptides may be used.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a functional block diagram of an exemplary system suitable for implementing the systems and methods disclosed herein.

FIG. 2 is a flowchart showing a method of a first embodiment of the invention.

FIG. 3 is a flowchart showing a method of a second embodiment of the invention.

### DETAILED DESCRIPTION

This disclosure presents various systems, components and methods for moving a liquid drug from a liquid reservoir in a drug delivery device to a patient interface, typically a needle or cannula. The embodiments described herein provide one or more advantages over conventional, prior art systems, components and methods, such as better blood glucose control, greater convenience, and less weight gain.

Various embodiments of the present invention include systems and methods for delivering a medication to a user using a wearable drug device (sometimes referred to herein as a “pod”), either autonomously, or in accordance with a wireless signal received from an electronic device. In various embodiments, the electronic device may be a user device comprising a smartphone, a smart watch, a smart necklace, a module attached to the wearable drug delivery device, or any other type or sort of electronic device that may be worn or carried on the body of the user and that executes an algorithm that computes the times and dosages of delivery of the medication. For example, the user device may execute an “artificial-pancreas” algorithm that computes the times and dosages of delivery of insulin. The user device may also be in communication with a sensor, such as a glucose sensor, that collects data on a physical attribute or condition of the user, such as a glucose level. The sensor may be disposed in or on the body of the user and may be part of the drug delivery device or may be a separate device. Alternatively, the drug delivery device may be in communication with the sensor in lieu of or in addition to the communication between the sensor and the user device. The communication may be direct (if, e.g., the sensor is integrated with or otherwise a part of the drug delivery device) or remote/wireless (if, e.g., the sensor is disposed in a different housing than the medical device). In these embodiments, the sensor and/or the drug delivery device contain computing hardware (e.g., a processor, memory, firmware, etc.) that executes some or all of the algorithm that computes the times and dosages of delivery of the medication.

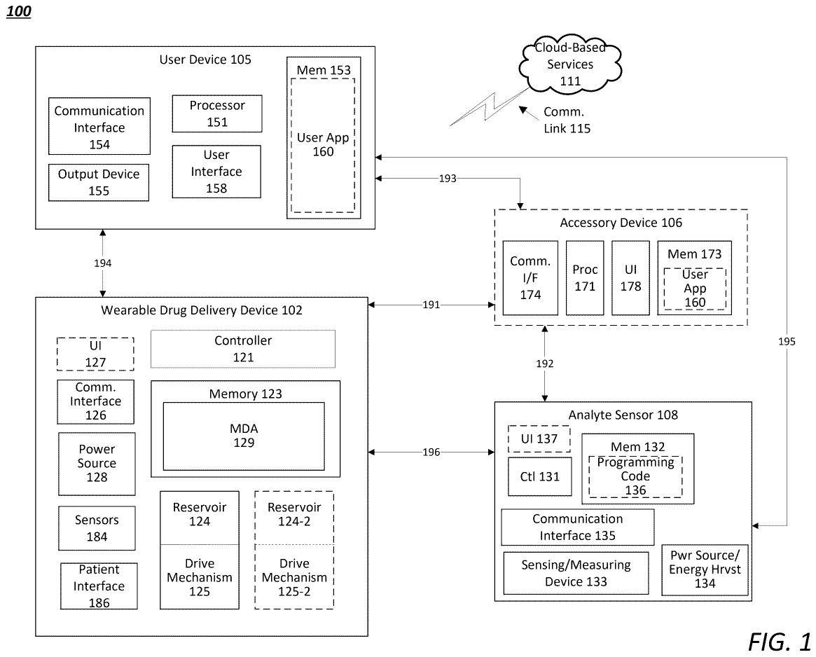

FIG. 1 illustrates a functional block diagram of an exemplary drug delivery system 100 suitable for implementing the systems and, methods described herein. The drug delivery system 100 may implement (and/or provide functionality for) a medication delivery algorithm to govern or control automated delivery of a drug or medication, such as insulin, to a user (e.g., to maintain euglycemia—a normal level of glucose in the blood). The drug delivery system 100 may be an automated drug delivery system that may include a drug delivery device 102, an analyte sensor 108, and a user device 105.

The drug delivery system 100, in an optional example, may also include an accessory device 106, such as a smartwatch, a personal assistant device or the like, which may communicate with the other components of system 100 via either a wired or wireless communication links 191-193.

The user device 105 may be a computing device such as a smartphone, a tablet, a personal diabetes management (PDM) device, a dedicated diabetes therapy management device, or the like. In an example, user device 105 may include a processor 151, device memory 153, a user interface 158, and a communication interface 154. The user device 105 may also contain analog and/or digital circuitry that may be implemented as a processor 151 for executing processes based on programming code stored in device memory 153, such as user application 160 to manage a user's blood glucose levels and for controlling the delivery of the drug, medication, or therapeutic agent to the user, as well for providing other functions, such as calculating carbohydrate-compensation dosage, a correction bolus dosage and the like as discussed below. The user device 105 may be used to program, adjust settings, and/or control operation of the drug delivery device 102 and/or the analyte sensor 103 as well as the optional smart accessory device 106.

The processor 151 may also be configured to execute programming code stored in device memory 153, such as the user app 160. The user app 160 may be a computer application that is operable to deliver a drug based on information received from the analyte sensor 103, the cloud-based services 111 and/or the user device 105 or optional accessory device 106. The memory 153 may also store programming code to, for example, operate the user interface 158 (e.g., a touchscreen device, a camera or the like), the communication interface 154 and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 is an insulin delivery application, the processor 151 is also configured to execute a diabetes treatment plan (which may be stored in a memory) that is managed by user app 160. In addition to the functions mentioned above, when user app 160 is an insulin delivery application, it may further provide functionality to determine a carbohydrate-compensation dosage, a correction bolus dosage and determine a basal dosage according to a diabetes treatment plan. In addition, as an insulin delivery application, user app 160 provides functionality to output signals to the drug delivery device 102 via communications interface 154 to deliver the determined bolus and basal dosages.

The communication interface 154 may include one or more transceivers that operate according to one or more radio-frequency protocols. In one embodiment, the transceivers may comprise a cellular transceiver and a Bluetooth® transceiver. The communication interface 154 may be configured to receive and transmit signals containing information usable by user app 160.

User device 105 may be further provided with one or more output devices 155 which may be, for example, a speaker or a vibration transducer, to provide various signals to the user.

An exemplary embodiment of the drug delivery device 102 may include a reservoir 124 and drive mechanism 125, which are controllable by controller 121, executing a medication delivery algorithm (MDA) 129 stored in memory 123. Alternatively, controller 121 may act to control reservoir 124 and drive mechanism 125 based on signals received from user app 160 executing on a user device 105 and communicated to drug delivery device 102 via communication link 194.

Drug delivery device 102 may further include a user interface 127, a patient interface 186, a communication interface 126, device sensors 184 and a power source 128.

In an alternate embodiment, drug delivery device 102 may also include an optional second reservoir 124-2 and second drive mechanism 125-2 which enables the independent delivery of two different liquid drugs (i.e., not as a co-formulation in the same reservoir). As an example, reservoir 124 may be filled with insulin, while reservoir 124-2 may be filled with Pramlintide or GLP-1. In some embodiments, each of reservoirs 124, 124-2 may be configured with a separate drive mechanism 125, 125-2, respectively, which may be separately controllable by controller 121 under the direction of MDA 129. Both reservoirs 124, 124-2 may be connected to a common patient interface 186.

Drug delivery device 102 may be optionally configured with a user interface 127 providing a means for receiving input from the user and a means for outputting information to the user. User interface 127 may include, for example, light-emitting diodes, buttons on a housing of the drug delivery device 102, a sound transducer, a micro-display, a microphone, an accelerometer for detecting motions of the device of user gestures (e.g., tapping on a housing of the device) or any other type of interface device that is configured to allow a user to enter information and/or allow the drug delivery device 102 to output information for presentation to the user (e.g., alarm signals or the like).

The drug delivery device 102 includes a patient interface 186 for interfacing with the user to deliver the liquid drug. Patient interface may be, for example, a needle or cannula for delivering the drug into the body of the user (which may be done subcutaneously, intraperitoneally, or intravenously). Drug delivery device 102 further includes a means for inserting the patient interface 186 into the body of the user which may comprise, in one embodiment, an actuator that inserts the needle/cannula under the skin of the user and thereafter retracts the needle, leaving the cannula in place.

In one embodiment, the drug delivery device 102 includes a communication interface 126, which may be a transceiver that operates according to one or more radio-frequency protocols, such as Bluetooth, Wi-Fi, near-field communication, cellular, or the like. The controller 121 may, for example, communicate with user device 105 and an analyte sensor 108 via the communication interface 126.

In some embodiments, drug delivery device 102 may be provided with one or more sensors 184. The sensors 184 may include one or more of a pressure sensor, a power sensor, or the like that are communicatively coupled to the controller 121 and provide various signals. For example, a pressure sensor may be configured to provide an indication of the fluid pressure detected in a fluid pathway between the patient interface 186 and reservoir 124. The pressure sensor may be coupled to or integral with the actuator for inserting the patient interface 186 into the user. In an example, the controller 121 may be operable to determine a rate of drug infusion based on the indication of the fluid pressure. The rate of drug infusion may be compared to an infusion rate threshold, and the comparison result may be usable in determining an amount of insulin onboard (IOB) or a total daily insulin (TDI) amount.

Drug delivery device 102 further includes a power source 128, such as a battery, a piezoelectric device, an energy harvesting device, or the like, for supplying electrical power to controller 121, memory 123, drive mechanisms 125 and/or other components of the drug delivery device 102.

The communication link 115 that couples the cloud-based services 111 to the respective devices 102, 105, 106, 108 of system 100 may be a cellular link, a Wi-Fi link, a Bluetooth link, or a combination thereof. Services provided by cloud-based services 111 may include data storage that stores anonymized data, such as blood glucose measurement values, historical IOB or TDI, prior carbohydrate-compensation dosage, and other forms of data. In addition, the cloud-based services 111 may process the anonymized data from multiple users to provide generalized information related to TDI, insulin sensitivity, IOB and the like.

The wireless communication links 191-196 may be any type of wireless link operating using known wireless communication standards or proprietary standards. As an example, the wireless communication links 191-196 may provide communication links based on Bluetooth®, Zigbee®, Wi-Fi, a near-field communication standard, a cellular standard, or any other wireless protocol via the respective communication interfaces 154, 174, 126 and 135.

The drug delivery device 102 may be configured to perform and execute processes required to deliver doses of the medication to the user without input from the user device 105 or the optional accessory device 106. As explained in more detail, MDA 129 may be operable, for example, to determine an amount of insulin to be delivered, IOB, insulin remaining, and the like and to cause controller 121 to control drive mechanism 125 to deliver the medication from reservoir 124. MDA 129 may take as input data received from the analyte sensor 108 or from user app 160.

The reservoirs 124, 124-2 may be configured to store drugs, medications or therapeutic agents suitable for automated delivery, such as insulin, Pramlintide, GLP-1, co-formulations of insulin and GLP-1, morphine, blood pressure medicines, chemotherapy drugs, fertility drugs or the like.

The drug delivery device 102 may be attached to the body of a user, such as a patient or diabetic, at an attachment location and may deliver any therapeutic agent, including any drug or medicine, such as insulin, a co-formulation as described above, or the like, to a user at or around the attachment location. A surface of the drug delivery device 102 may include an adhesive to facilitate attachment to the skin of a user.

When configured to communicate with an external device, such as the user device 105 or the analyte sensor 108, the drug delivery device 102 may receive signals via link 194 from the user device 105 or via link 196 from the analyte sensor 108. The controller 121 of the drug delivery device 102 may receive and process the signals from the respective external devices as well as implementing delivery of a drug to the user according to a diabetes treatment plan or other drug delivery regimen, implemented by MDA 129 or user application 160.

In an operational example, the controller 121, when executing MDA 129 may generate and output a control signal operable to actuate the drive mechanism 125 to deliver a carbohydrate-compensation dosage of insulin, a correction bolus, a revised basal dosage, co-formulations of various liquid drugs, or the like.

The accessory device 106 may be, for example, an Apple Watch®, other wearable smart device, including eyeglasses, smart jewelry, a global positioning system-enabled wearable, a wearable fitness device, smart clothing, or the like. Similar to user device 105, the accessory device 106 may also be configured to perform various functions including controlling the drug delivery device 102. For example, the accessory device 106 may include a communication interface 174, a processor 171, a user interface 178 and a memory 173. The user interface 178 may be a graphical user interface presented on a touchscreen display of the smart accessory device 106. The memory 173 may store programming code to operate different functions of the smart accessory device 106 as well as an instance of the user app 160, or a pared-down versions of user app 160 with reduced functionality.

The analyte sensor 108 may include a controller 131, a memory 132, a sensing/measuring device 133, an optional user interface 137, a power source/energy harvesting circuitry 134, and a communication interface 135. The analyte sensor 108 may be a continuous glucose monitor (CGM) and may be communicatively coupled to the processor 151 of the management device 105 or controller 121 of the drug delivery device 102. The memory 132 may be configured to store information and programming code 136.

The analyte sensor 108 may be configured to detect one or more different analytes, such as glucose, lactate, ketones, uric acid, sodium, potassium, alcohol levels or the like, and output results of the detections, such as measurement values or the like. The analyte sensor 108 may, in an exemplary embodiment, be configured as a CGM to measure blood glucose values at a predetermined time intervals, such as every 5 minutes, or the like. The communication interface 135 of analyte sensor 108 may have circuitry that operates as a transceiver for communicating the measured blood glucose values to the user device 105 over a wireless link 195 or with drug delivery device 102 over the wireless communication link 108. While referred to herein as an analyte sensor 108, the sensing/measuring device 133 of the analyte sensor 108 may include one or more additional sensing elements, such as a glucose measurement element, a heart rate monitor, a pressure sensor, or the like. The controller 131 may include discrete, specialized logic and/or components, an application-specific integrated circuit, a microcontroller or processor that executes software instructions, firmware, programming instructions stored in memory (such as memory 132), or any combination thereof.

Similar to the controller 221 of drug delivery device 102, the controller 131 of the analyte sensor 108 may be operable to perform many functions. For example, the controller 131 may be configured by programming code 136 to manage the collection and analysis of data detected by the sensing and measuring device 133.

Although the analyte sensor 108 is depicted in FIG. 1 as separate from the drug delivery device 102, in various embodiments, the analyte sensor 108 and drug delivery device 102 may be incorporated into the same unit. That is, in various examples, the analyte sensor 108 may be a part of and integral with the drug delivery device 102 and contained within the same housing as the drug delivery device 102. In such an embodiment, the controller 221 may be able to implement the functions required for the proper delivery of the medication alone without any external inputs from user device 105, the cloud-based services 111, another sensor (not shown), the optional accessory device 106, or the like.

The user app 160 (or MDA 129) may provide periodic insulin micro-boluses based upon the predicted glucose over a 60-minute prediction horizon. The user app 160 or MDA 129 may compensate for missed meal boluses and mitigate prolonged hyperglycemia. The user app 160 or MDA 129 may use a control-to-target strategy that attempts to achieve and maintain a set target glucose value, thereby reducing the duration of prolonged hyperglycemia and hypoglycemia.

The user app 160 implements a graphical user interface that is the primary interface with the user and may be used to control, activate or de-activate drug delivery device 102, program basal and bolus calculator settings for a manual mode as well as program settings specific for an automated mode (hybrid closed-loop or closed-loop).

In manual mode, user app 160 will deliver insulin at programmed basal rates and bolus amounts with the option to set temporary basal profiles. The controller 121 will also have the ability to function as a sensor-augmented pump in manual mode, using sensor glucose data provided by the analyte sensor 108 to populate the bolus calculator.

In automated mode, the user app 160 supports the use of multiple target blood glucose values. For example, in one embodiment, target blood glucose values can range from 110-150 mg/dL, in 10 mg/dL increments, in 5 mg/dL increments, or other increments, but preferably 10 mg/dL increments. The experience for the user will reflect current setup flows whereby the healthcare provider assists the user to program basal rates, glucose targets and bolus calculator settings. These, in turn, will inform user app 160 or MDA 120 of insulin dosing parameters. The insulin dosing parameters will be adapted over time based on the total daily insulin (TDI) delivered during each use of drug delivery device 102. A temporary hypoglycemia protection mode may be implemented by the user for various time durations in automated mode. With hypoglycemia protection mode, the algorithm reduces insulin delivery and is intended for use over temporary durations when insulin sensitivity is expected to be higher, such as during exercise.

User app 160, allows the use of large text, graphics, and on-screen instructions to prompt the user through the set-up processes and the use of system 100. It will also be used to program the user's custom basal insulin delivery profile, check the status, of drug delivery device 102, initiate bolus doses of insulin, make changes to a patient's insulin delivery profile, handle system alerts and alarms, and allow the user to switch between automated mode and manual mode.

In some embodiments, user device 105 and the analyte sensor 108 may not communicate directly with one another. Instead, data (e.g., blood glucose readings) from analyte sensor may be communicated to drug delivery device 102 via link 196 and then relayed to user device 105 via link 194. In some embodiments, to enable communication between analyte sensor 108 and user device 105, the serial number of the analyte sensor must be entered into user app 160.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value and insulin on board (IOB). IOB is estimated by user app 160 taking into account any manual bolus and insulin delivered by the algorithm.

Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In a first embodiment of the invention, drug delivery system 100 is configured to detect when the user has ingested a meal and to deliver a dose of Pramlintide in response to the meal ingestion. The dose of Pramlintide delays gastric emptying when injected into the user, resulting in a reduction in the peak, but a broadening of the breath, of post-prandial hyperglycemia in people with type I diabetes.

There is no specific dosage that is recommended for specific meal ingestion (i.e., a calculation of a dose of Pramlintide based on the carbohydrate profile of the ingested meal). Rather, there is a fixed dosage recommended by clinicians for a fixed outcome in the user's glucose excursions. Specifically, a 30-60 μg per meal dose of Pramlintide, that is provided at the time of the meal, results in a mean glucose reduction of approximately 14 mg/dL in the user's blood glucose readings.

MDA 129 and user app 160 are designed to be limited in the aggressiveness with which the drug delivery system 100 can act against minor deviations in the user's glucose levels. This may lead to the user experiencing an extended period of hyperglycemia if there is no manual compensation for the meal (e.g., a bolus injection). Thus, providing the Pramlintide when a meal is detected allows a less-aggressive insulin delivery action of MDA 129 or user app 160 to address the glucose excursions of the user due to the ingestion of the meal. As such, no adjustments to the parameters of MDA 129 or user app 160 is necessary to address the ingestion of the meal.

In some embodiments of drug delivery system 100, analyte sensor 108 may deliver information to drug delivery device 102 or user device 105 regarding the user's current glucose level every five minutes. Other embodiments, the provision of the glucose levels may be provided using different time intervals, or, when requested by MDA 129 or user app 160. Analyte sensor 108 may provide the glucose readings to drug delivery device 102 via communication link 196 or to user device 105 via communications link 195.

To determine when to provide the user with the dose of Pramlintide, it is first necessary that drug delivery system 100 be able to detect when the user has ingested a meal. This is accomplished via an analysis of the trends of the blood glucose readings received from analyte sensor 108. A rapid and persistent increase in the user's glucose level readings or changes in the user's mean glucose readings over time may thus be interpreted to indicate a high probability that the user has ingested a meal. In certain embodiments of the invention, the probability of meal ingestion may be calculated immediately upon receipt of each new current glucose reading from analyte sensor 108.

In a first aspect of the invention, if the user's glucose concentration increases by more than 120 mg/dL (20 mg/dL/cycle) in the last 30 minutes, it is assumed that there is a high probability (P_{meal,trend}) that the user has ingested a meal. Thus:

$${P}_{meal , trend} ( i ) = max ( 0 , \frac{C G M ( i ) - C G M ( i - t )}{y} ) \qquad ( 1 )$$

where:
i is the current cycle (i.e., the current 5 minute period during which a new glucose reading has been received);
CGM(i) is the glucose reading fir the current cycle;
t is the number of cycles over which the trend is being analyzed; (e.g., t=6 for a 30 m interval); and
y is the threshold for determining if the meal has been ingested (e.g., 120 mg/dL).

Note that, in embodiments of the invention where another time intervals for the receipt of the blood glucose level readings are used, or wherein a different threshold for determining the ingestion of a meal is used, the constants in the Eq. (1) will change.

In a second aspect of the invention, if the user's blood glucose level is more than 90 mg/dL above a target, this is indicative of the hybrid probability that the user has ingested a meal. Thus:

$${P}_{meal , val} ( i ) = max ( 0 , \frac{C G M ( i ) - S P ( i )}{y} ) \qquad ( 2 )$$

where:
i is the current cycle (i.e., the current 5 minute period during which a new glucose reading has been received);
CGM(i) is the glucose reading for the current cycle;
SP(i) is the target glucose reading for the current cycle; and
y is the threshold for determining if a meal has been ingested (e.g., 90 mg/dL).

In a third aspect of the invention, the probability of a meal ingestion may be high if the user's mean glucose in the last 30 minutes is at least 60 mg/dL higher than the user's mean glucose in the 30 minutes prior to the current 30 minutes set. Thus:

$${P}_{meal , mean} ( i ) = max ( 0 , \frac{\frac{{\sum }_{j = 1}^{t} G ( i - j )}{t} - \frac{{\sum }_{j = 1}^{t} G ( i - j - t )}{t}}{y} ) \qquad ( 3 )$$

where:
i is the current cycle (i.e., the current 5 minute period during which a new glucose reading has been received);

$$\frac{{\sum }_{j = 1}^{t} G ( i - j )}{t}$$

is the user's mean glucose reading for the past t cycles;

$$\frac{{\sum }_{j = 1}^{t} G ( i - j - t )}{t}$$

is the user's mean glucose reading for t cycles before the current t cycles; and
y is the threshold for determining if a meal has been ingested (e.g., 60 mg/dL).

While either of the first three methods for determining the probability that a meal has been ingested may be used, in preferred embodiments of the invention, all three methods may be used in an average of the three taken. Thus:

$${P}_{m e a l} ( i ) = \frac{{P}_{m eal , trend} ( i ) + {P}_{m e al , val} ( i ) + {P}_{m e a l , m e a n} ( i )}{3} \qquad ( 4 )$$

where:
i is the current cycle (i.e., the current 5 minute period during which a new glucose reading has been received); and
Pₘₑₐₗ(i) represents a probability that the user has taken a meal in the last t cycles (e.g., t=6 represents 30 min).

In this embodiment, if Pₘₑₐₗ(i) is above a certain threshold (e.g., 0.5), the system may determine that a meal has been ingested and may deliver a fixed dose of Pramlintide to the user. In various embodiments of the invention, the quantity of Pramlintide delivered may be between 30 μg and 60 μg, however, in preferred embodiments, a 30 μg dose is provided. Also, in preferred embodiments of the invention, it is desirable that the dose of Pramlintide only be provided once per meal. Thus, if the dose of Pramlintide has been delivered within a predetermined past number of cycles (e.g., 18 cycles, indicating 90 min), the Pramlintide will not be delivered again until expiration of this time period and a further indication that the user's blood glucose readings indicate a probability that a meal has been ingested.

In preferred embodiments of the invention, drug delivery device 102 is provided with two reservoirs 124 and 124-2, as shown in FIG. 1, each with its own respective drive mechanism 125, 125-2. In this embodiment, one reservoir 124 may be filled with insulin while the second reservoir, 124-2 is filled with pramlintide. As such, quantities of insulin and Pramlintide made be delivered independently of each other. In alternate embodiments, the invention may be implemented using two single-reservoir drug delivery devices, one having a reservoir dispensing insulin and one having the reservoir dispensing Pramlintide.

Once the ingestion of a meal has been detected and the dose of Pramlintide delivered to the user, insulin may be administered in accordance with MDA 129 or user app 160, which may eliminate the need for a user to manually administer a bolus dose of insulin in response to the ingestion of the meal. The reduction in the mean glucose levels of the user during the post-prandial period means MDA 129 or user app 160, with limited aggressiveness, may be able to address the post-prandial rise in the user's glucose levels without the user experiencing extended period of hypoglycemia.

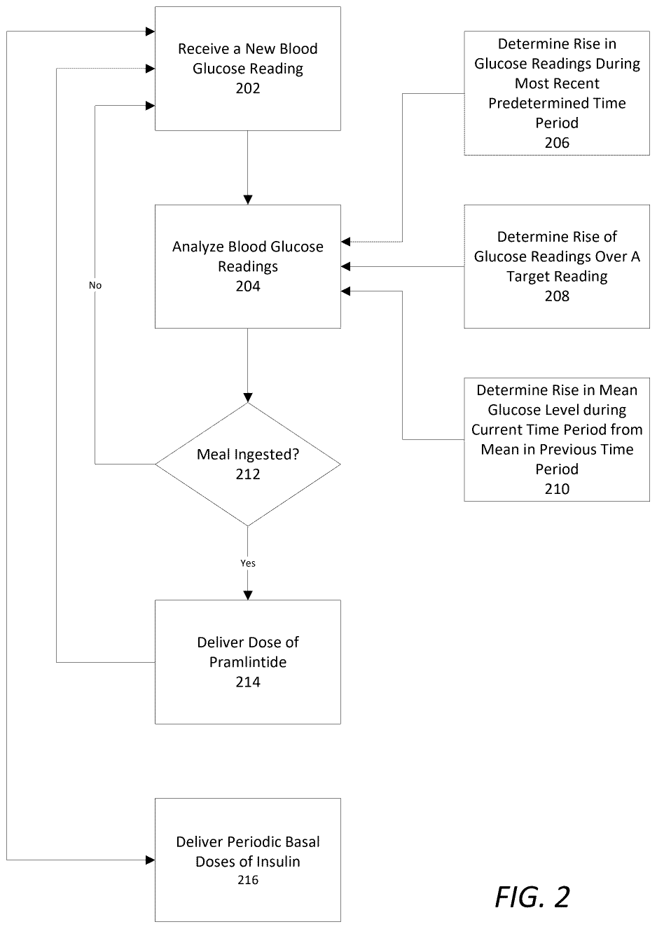

FIG. 2 is a flowchart showing flow of a method in accordance with the first embodiment of the invention. At 202, the drug delivery system 100 receives periodically receives a blood glucose reading of the user. For example, in certain embodiments, the drug delivery device 102 will receive blood close readings directly from the analyte sensor 108 via communication link 196. Based on the received blood glucose readings, MDA 129 delivers a periodic basal dose of insulin at 216 and returns to 2022 await a new blood glucose reading.

In addition to the periodic basal dose of insulin, MDA 120 and also determines if a dose of pramlintide should be delivered to the user in response to the user to ingesting a meal. At 204, MDA 129 analyzes the blood glucose readings via one of the methods previously described to determine if the user has ingested a meal. At 206, MDA 129 may determine that the user's blood glucose concentration has increased more than a predetermined amount in most recent period of time, for example, an increase of more than 120 mg/dL during the past 30 minutes, in accordance with the Eq. (1). At 208, MDA 129 may determine that the user's blood current blood glucose reading is a predetermined amount above a target blood glucose reading, for example, 90 mg/dL above the target, in accordance with Eq. (2). At 210, MDA 129 may determine that the user's mean glucose during a most recent predetermined period of time is at least a predetermined amount higher than the user's mean glucose in a predetermined time period immediately preceding the most recent predetermined time period. MDA 129 may determine that use mean glucose in the past 30 minutes is at least 60 md/dL higher than the user's mean glucose in the 30 minutes prior to the current 30 minutes. In accordance with Eq. (3). Either one of Eqs. (1)-(3) may be used independently or in any combination to determine the probability that the user has ingested a meal. In yet another embodiment, all three of Eqs. (1)-(3) may be calculated and averaged to produce the probability that the user has ingested a meal. Other methods or criteria may also be used to determine if the user has ingested a meal.

At 212, if the user has ingested a meal, a dose of pramlintide is delivered at 214 and control is returned to 202 where MDA 129 receives further periodic glucose readings. The method shown in FIG. 2 may be repeated for each cycle, that is, each time a new blood glucose reading is received.

In a second embodiment of the invention, drug delivery device 102 is provided with a single reservoir 124 containing a co-formulation of insulin and Pramlintide. The co-formulation is incorporated into the calculation of basal doses of insulin by either MDA 129 or user app 160.

In one embodiment, wherein drug delivery device 102 stores enough insulin in reservoir 124 for three days (200 units). In this embodiment, assuming an average dose of Pramlintide of 45 μg/meal, and 6 meal/day, a three day supply of Pramlintide should require approximately

$$810 μg ( 45 \frac{μg}{meal} \times 6 \frac{meal}{day} \times 3 days = 810 μg = 81 mg )$$

of Pramlintide to be dissolved in the 200 units of insulin. In this embodiment, the amount of Pramlintide that is delivered per 0.05 units of insulin (i.e., in some embodiments, the smallest amount of insulin that can be delivered by drug delivery device 102) is

$$\text{.2025} μg ( \frac{810 μg}{200 units} \times 0.05 units = \text{.2025} μg ) .$$

As previously stated with respect to the first embodiment, the administration of Pramlintide has the capacity to reduce the user's mean glucose by 14 mg/dL. Given a typical target glucose of 120 mg/dL, this represents an approximately 11.7% reduction in the user's insulin needs. This can be translated into an estimated adjustment factor for MDA 129 by relating the daily delivery within 11.7% impact in the user's glucose concentration:

$${F}_{p r} = \frac{0.117}{0.27} = \frac{0.43}{{mg}_{p r}} \qquad ( 5 )$$

The adjustment factor can then be used to calculate the total pramlintide delivery during the previous 24 hours, which can be used to modify the actions of MDA 129 or user app 160:

$${AID}_{p r} ( i ) = 0.43 \cdot \frac{{\sum }_{j = 1}^{288} I ( j )}{0.05} \cdot 0.0002025 \qquad ( 6 )$$

where:
i is the current cycle; and
288 is a constant indicating the number of cycles during the past 24 hours, assuming a 5 minute cycle.

MDA 129 or user app 160 uses a parameter known as the Q:R ratio to determine the aggressiveness of MDA 129. A reduction the Q:R ratio results in an increase in the aggressiveness of the algorithm. Administering a co-formulation of Pramlintide affects the Q term of the Q:R ratio in the following manner:

$${Q}_{n e w} = {{Q}_{B a s e} ( 1 - {AID}_{p r} )}^{2} \qquad ( 7 )$$

In addition, MDA 129 also utilizes a maximum integral constraint which limits or constrains the response of the MDA 129 to detected excursions in the user's blood glucose level. As such, a reduction in the maximum integral constraint results in a reduction in the aggressiveness of MDA 129. A new maximum integral constraint can be calculated by the equation:

$${U}_{max , integral new} = {U}_{max , integral} ( 1 - {AID}_{pr} ) \qquad ( 8 )$$

In this embodiment of the invention, the single reservoir of the drug delivery device 102 contains a co-formulation of Pramlintide and insulin. In one embodiment, the relative concentrations of the insulin and Pramlintide in the co-formulation can be made in accordance with the above calculations, which uses certain assumptions. For example, the calculations assume 6 meals per day (i.e., 6 bolus doses of insulin administered by the user each day) and the dosage of the pramlintide to be 45 μg/meal. In other embodiments of the invention, other assumptions may be made. For example, the dosage of the Pramlintide on a per-meal basis may be altered.

The reduction in the user's target glucose allows parameters of the MDA 129 to be altered. In one instance, an increase in the aggressiveness of MDA 129 is acceptable given the reduced concern of extreme hypoglycemia resulting in increased risk of over delivery. In another instance, a reduction in the integral insulin constraint may be possible, given extended period of each hyperglycemic event.

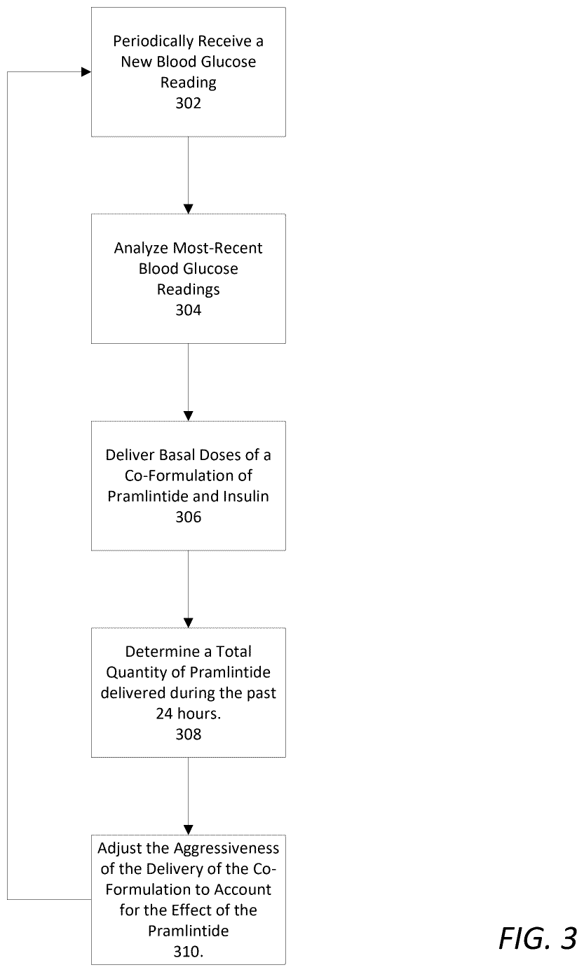

FIG. 3 is a flowchart showing flow of a method in accordance with the second embodiment of the invention. At 302, MDA 129 periodically receives a new blood glucose readings from sensor 108. At 304, the user's most-recent blood glucose readings from a most-recent predetermined period of time are analyzed to determine if any adjustment is required to the basal rate at which basal doses of insulin are being delivered to the user. At 306 basal doses of a co-formulation of pramlintide and insulin are delivered to the user. At 308, a calculation is made to determine the total quantity of pramlintide which is been delivered to the user during a most recent predetermined period of time, for example, 24 hours. Based on the total quantity of pramlintide it delivered during the predetermined period of time, the aggressiveness of the response of MDA 129 is adjusted. For example, if increased quantities of pramlintide have been delivered, MDA 129 may be allowed to provide a more aggressive response to excursions in the user's blood glucose level, while the total amount of insulin per period of time permitted to be delivered by MDA 129 may be decreased. The method and returns to 302 and repeats.

The examples above refer to a set or pre-determined amount of a second drug, such as pramlintide, having been co-formulated with insulin. As users incorporate a co-formulation of insulin and pramlintide, GLP-1 or other drugs into their regimen, the co-formulation may initially be delivered with a fixed amount of each additional drug in the reservoir with insulin. As this provides a fixed amount of pramlintide (or other drug) when delivered into the reservoir with insulin, or a fixed ratio of pramlintide and insulin when co-formulated with a set amount of insulin, this amount or this ratio may not be ideal for all users, who may vary in their needs for pramlintide that are independent from their insulin needs.

In a further exemplary embodiment, a method is disclosed for an insulin pump system to review the user's previously recorded daily insulin needs against their most recent insulin delivery history, and modify the concentration of pramlintide, GLP-1 or other drug, that is co-formulated with insulin for the next iteration of a pump reservoir refill. This can be done automatically via an auto reservoir fill mechanism or manually by the user based on a suggestion from the insulin pump system.

Pramlintide, GLP-1 and/or other co-formulated drugs with insulin may have fixed or personalized daily doses that may be recommended by a physician, which may be independent from the user's insulin needs. However, in co-formulated drugs, the amount of insulin the user receives per day may impact the amount of each co-formulated drug that the users may also receive.

In an exemplary embodiment, an automated or manual preparation of co-formulated drugs with insulin may initially apply a fixed amount of such drugs per one full reservoir, such as 200 U of insulin (e.g., 3 mg of GLP1 per 200 U).

This initial dose, D_{co}, can subsequently be adjusted based on the expected daily dose of each drug against the changes in the user's total insulin needs. Specifically, the final amount of each co-formulated drug that can be incorporated into the user's new reservoir fill can be adjusted as follows:

$${D}_{c o} ( j ) = {D}_{c o} ( 1 ) \cdot \frac{TDI ( 1 )}{TDI ( j )} \qquad ( 9 )$$

where:
TDI(1) is the Total Daily Insulin (TDI) of the user during which the co-formulated drug was prescribed by the physician; and
TDI(j) is the user's adapted TDI for the next cycle when the user's TDI is reassessed, which may be based on the following exemplary weighting equation:

$$TDI ( j ) = ( 1 - 0.2 \cdot {N}_{d a y s} ) TDI ( j - 1 ) + 0.2 \cdot {N}_{d a y s} \sum I ( j - 1 ) \qquad ( 10 )$$

Note that this exemplary embodiment may reduce the suggested amount of co-formulated drug to be included with the new insulin reservoir. This is the case if, to maintain the same amount of total co-formulated drug dose to the user each day, as the user's daily insulin needs increase, the amount of drug being delivered per insulin dose must be decreased—which is achieved by reducing the overall amount of co-formulated drug that is being incorporated into the insulin reservoir. This results in a reduction in the concentration of the co-formulated drug within the insulin reservoir, and thus a reduction in the amount of the co-formulated drug being delivered per insulin dose. In cases where the user's adapted TDI for a next cycle (e.g., a three-day cycle of wearing an insulin pump) decreases, then D_{co}(j), or the amount of the co-formulated drug for the next cycle or the new reservoir, may increase. To receive the same amount of secondary drug (e.g., GLP-1 or pramlintide) as the user receives less insulin, the concentration of the secondary drug in the insulin must be increased. In this manner, the ratio of the secondary drug and insulin may be adjusted to account for changes in the user's insulin needs.

After the modified amount of the co-formulated drug for the next cycle for the new reservoir is calculated (D_{co}(j)), it may be output to the user with instructions or a recommendation for how much to insert into the next reservoir. Alternately, this modified amount can inform an automated drug co-formulation device that can automate the dosage of the appropriate amount of co-formulated GLP-1 or other drug when filling each new insulin reservoir. For example, the system may output to the user via a user interface 158 of user device 105 to insert or co-formulate 6.0 mg of GLP-1 per 200 U of insulin, instead of the previous 3.0 mg of GLP-1 per 200 U of insulin, which corresponds to a 100% increase in the concentration of GLP-1 within the 200 U reservoir. This recommendation would be provided if the user's current total daily insulin need has been reduced to 50% of the original total daily insulin in which the original daily GLP-1 dose was prescribed by the physician, meaning each subsequent insulin dose must deliver double the amount of GLP-1 that was first delivered within the same insulin dose, to provide the same total amount of GLP-1 to the user per day. In this manner, concentrations of the secondary drug (e.g., GLP-1 or pramlintide) may be adjusted as the user's insulin needs change.

The following examples pertain to various embodiments of the systems and methods disclosed herein for implementation of an automatic drug delivery system dispensing with Pramlintide and insulin.

Example 1 is a system implementing a first embodiment of the invention comprising one or more drug delivery devices configured to deliver doses of insulin and pramlintide and an algorithm for determining the timing and quantity of the delivery of the insulin and the pramlintide independently of each other.

Example 2 is an extension of Example 1, or any other example disclosed herein, wherein the algorithm determines when the user has ingested a meal.

Example 3 is an extension of Example 2, or any other example disclosed herein, wherein the system further comprises a sensor for periodically providing blood glucose level readings of the user to the algorithm.

Example 4 is an extension of Example 3, or any other example disclosed herein, wherein the algorithm directs one of the drug delivery devices to deliver a dose of pramlintide in response to the ingestion of a meal.

Example 5 is an extension of Example 1, or any other example disclosed herein, wherein a single drug to delivery device having two reservoirs, one containing insulin in one containing pramlintide is used.

Example 6 is an extension of Example 3, or any other example disclosed herein, wherein it is determined that the user has ingested a meal based on one or more of three separate criteria: a predetermined rise in the glucose readings of the user during most recent predetermined time; a predetermined rise in the glucose readings over a target glucose reading; or a predetermined rise in the mean glucose level of the user over a predetermined time period over the mean glucose level of the user during a preceding predetermined time period.

Example 7, is an extension of Example 3, or any other example disclosed herein, wherein it is determined that the user has ingested a meal based on an average of one or more of the three separate criteria listed in Example 6.

Example 8 is a method implementing a first embodiment of the invention comprising determining that a user has ingested a meal and automatically delivering a dose of pramlintide to the user.

Example 9 is an extension of Example 8, or any other example disclosed herein, wherein the method further comprises receiving periodic blood glucose readings and analyzing a plurality of the most recent blood glucose readings to determine an ingestion of a meal.

Example 10 is an extension of Example 8, or any other example disclosed herein, wherein the method further comprises delivering basal doses of insulin to the user based on the periodic blood glucose readings.

Example 11 is an extension of Example 9, or any other example disclosed herein, wherein the method further comprises determining that the user has ingested a meal based on one or more of three separate criteria: a predetermined rise in the glucose readings of the user during most recent predetermined time; a predetermined rise in the glucose readings over a target glucose reading; or a predetermined rise in the mean glucose level of the user over a predetermined time period over the mean glucose level of the user during a preceding predetermined time period.

Example 12 is an extension of Example 8, or any other example disclosed herein, wherein the method further comprises determining that the user has ingested a meal based on an average of one or more of the three separate criteria listed in Example 11.

Example 13 is a system implementing a second embodiment of the invention comprising a drug delivery device configured to deliver co-formulated doses of insulin and pramlintide and an algorithm for determining the timing and quantity of the delivery of the co-formulation.

Example 14 is an extension of Example 13, or any other example disclosed herein, wherein the algorithm calculates the total quantity of pramlintide delivered during a most recent predetermined period of time and adjust the delivery of basal doses based on the calculation

Example 15 is an extension of Example 14, or any other example disclosed herein, wherein the predetermined period of time 24 hours.

Example 16 is extension of Example 14, or any other example disclosed herein, wherein the over them provides a more aggressive response to blood glucose excursions based on a higher quantity of pramlintide being delivered during the most recent predetermined period of time.

Example 17 is an extension of Example 14, or any other example disclosed herein, wherein the algorithm decreases the total quantity of the co-formulation delivered within a certain period of time based on the quantity of pramlintide being delivered during the most recent predetermined period of time.

Example 18 is an extension of Example 13, or any other example disclosed herein, wherein the algorithm controls delivery of basal doses of the co-formulation based on the periodic blood glucose readings.

Example 19 is a method implementing a second embodiment of the invention comprising: calculating periodic basal doses of the co-formulation of pramlintide insulin, calculating a total amount of pramlintide delivered to the user during the most recent predetermined period of time and adjusting the periodic basal doses based on the amount of pramlintide delivered to the user

Example 20 is an extension of Example 19, or any other example disclosed herein, the method further comprising allowing a higher dose of the co-formulation to be delivered to the user in response to sustained increases in the blood glucose readings of the user.

Example 21 is an extension of Example 19, or any other example disclosed herein, the method further comprising reducing the total amount of the co-formulation to be delivered to the user based on a sustained increase in blood glucose readings of the user.

To those skilled in the art to which the invention relates, many modifications and adaptations of the invention may be realized. Implementations provided herein, including sizes, shapes, ratings and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A system comprising:
one or more drug delivery devices configured to deliver doses of insulin and pramlintide to a user; and
an algorithm, implemented by software executed by a processor, to determine the timing and quantity of the delivery of the insulin and the pramlintide.

2. The system of claim 1 wherein the algorithm determines when the user has ingested a meal.

3. The system of claim 2 further comprising:
a sensor, for periodically providing blood glucose level of the user to the algorithm, wherein the determination that the user has ingested a meal is based on the periodic blood glucose levels.

4. The system of claim 3 wherein the algorithm directs one of the drug delivery devices to deliver a dose of pramlintide to the user when the algorithm determines that the user has ingested a meal.

5. The system of claim 1 wherein the one or more drug delivery devices comprises a single drug delivery device having two reservoirs, one reservoir containing insulin and the other reservoir containing pramlintide.

6. The system of claim 3 wherein the determination that the user has ingested a meal is based on one or more of: a predetermined rise in the glucose readings of the user during most recent predetermined time; a predetermined rise in the glucose readings over a target glucose reading; or a predetermined rise in the mean glucose level of the user over a predetermined time period over the mean glucose level of the user during a preceding predetermined time period.

7. The system of claim 3 wherein the determination that the user has ingested a meal is based on an average over a predetermined threshold of: a predetermined rise in the glucose readings of the user during most recent predetermined time; a predetermined rise in the glucose readings over a target glucose reading; and a predetermined rise in the mean glucose level of the user over a predetermined time period over the mean glucose level of the user during a preceding predetermined time period.

8. A method comprising:
determining that a user of a drug delivery system has ingested a meal; and
automatically delivering a dose of pramlintide to the user using the system of claim 1.

9. The method of claim 8 further comprising:
receiving periodic blood glucose reading of the user; and
analyzing a plurality of the most recent blood glucose readings to determining that a user of a drug delivery system has ingested a meal.

10. The method of claim 8 further comprising:
delivering basal does of insulin to the user based on the periodic blood glucose readings.

11. The method of claim 9 further comprising:
determining that the user has ingested a meal is based on one or more of: a predetermined rise in the glucose readings of the user during most recent predetermined time; a predetermined rise in the glucose readings over a target glucose reading; or a predetermined rise in the mean glucose level of the user over a predetermined time period over the mean glucose level of the user during a preceding predetermined time period.

12. The method of claim 8 further comprising:
determining that the user has ingested a meal is based on an average over a predetermined threshold of: a predetermined rise in the glucose readings of the user during most recent predetermined time; a predetermined rise in the glucose readings over a target glucose reading; and a predetermined rise in the mean glucose level of the user over a predetermined time period over the mean glucose level of the user during a preceding predetermined time period.
